(19) United States
(12) Patent Application Publication
(10) Pub. No.: US 2005/0124837 A1
(43) Pub. Date: Jun. 9, 2005

(54) Preparation of alkenylaromatic compounds

(76) Inventors: Christian Walsdorff, Ludwigshafen (DE); Michael Baier, Mannheim (DE); Klaus Harth, Altleiningen (DE); Susanne Weber, Obrigheim (DE); Wilhelm Ruppel, Mannheim (DE); Gotz-Peter Schindler, Mannheim (DE)

Correspondence Address: Keil & Weinkauf, 1350 Connecticut Avenue NW, Washington, DC, 20036, US

(21) Appl. No.: 10/501,878

(22) PCT Filed: Jan. 20, 2003
(86) PCT No.: PCT/EP03/00489

(30) Foreign Application Priority Data
Jan. 23, 2002 (DE) 102 02 647.5

(51) Int. Cl. C07C 2/68

(52) U.S. Cl. 585/467

(57) ABSTRACT

A process for preparing alkenylaromatic compounds by reacting alkylaromatic compounds in the presence of steam and natural gas or methane over a suitable catalyst at a temperature of from 400 to 800° C. and a pressure of from 0.01 to 10 bar, wherein the molar ratio of steam to alkylaromatic compound is from 6.9:1 to 1:1 or the molar ratio of methane to alkylaromatic compound is from 0.1:1 to 8:1.

The present invention relates to a process for preparing alkenylaromatic compounds by reacting alkylaromatic compounds in the presence of steam and natural gas or methane over a suitable catalyst, wherein the molar ratio of steam to alkylaromatic compound is from 6.9:1 to 1:1.

U.S. Pat. No. 3,847,968 discloses a process for preparing styrene, which comprises replacing a portion of the steam added by a certain quantity of fuel gas, in particular having a high methane content. Steam consumption is somewhat reduced as a result.

DE-A-15 93 370 discloses a process for preparing styrene or divinylbenzene by dehydrogenation of appropriate ethylbenzenes at from 500 to 700° C., wherein the molar ratio of hydrocarbon to steam is from 2:1 to 20:1.

These processes, in particular with regard to specific steam consumption, i.e. the ratio of steam used to styrene produced, leave something to be desired.

It is an object of the present invention to remedy these disadvantages.

We have found that this object is achieved by a novel and improved process for preparing alkenylaromatic compounds by reacting alkylaromatic compounds in the presence of steam and natural gas or methane over a suitable catalyst at a temperature of from 400 to 800° C. and a pressure of from 0.01 to 10 bar, wherein the molar ratio of steam to alkylaromatic compound is from 6.9:1 to 1:1.

The process according to the invention may be carried out as follows:

Alkylaromatic compound, steam and natural gas or methane are passed batchwise or, preferably, continuously at a temperature of from 400 to 800° C., preferably from 450 to 700° C., more preferably from 500 to 650° C., and a pressure of from 0.01 to 10 bar, preferably from 0.05 to 2 bar, more preferably from 0.1 to 1 bar, in particular from 0.2 to 0.8 bar, over a suitable catalyst.

The molar ratio of steam to alkylaromatic compound is generally from 6.9:1 to 1:1, preferably from 5.95:1 to 2:1, more preferably from 5.9:1 to 2.5:1, in particular 5.5:1 to 3:1.

The molar ratio of natural gas or methane to alkylaromatic compound is generally from 0.1:1 to 8:1, preferably from 0.2:1 to 7:1, more preferably from 0.5:1 to 6:1, in particular from 0.7:1 to 5:1.

Useful natural gas comprises at least 90 vol %, preferably at least 92 vol %, more preferably at least 95 vol %, in particular at least 97 vol % of methane.

Useful alkylaromatic compounds include all aromatic and heteroaromatic alkyl compounds, and preference is given to those in which the alkyl radical is unbranched or branched and contains from two to six carbon atoms. Useful aromatic radicals include mono-, bi- or tricyclic, preferably mono- or bicyclic, more preferably monocyclic, aromatics. Examples include isopropylbenzene (cumene), ethylbenzene, 1,1-diphenylbenzene and 1,2-diphenylethane (bibenzyl), preferably isopropylbenzene (cumene), ethylbenzene and 1,1-diphenylbenzene, more preferably ethylbenzene. Useful heteroaromatic radicals include mono-, bi- or tricyclic, preferably mono- or bicyclic, more preferably monocyclic five-membered ring heteroaromatics having from one to three nitrogen atoms and/or an oxygen or sulfur atom, mono-, bi- or tricyclic, preferably mono- or bicyclic, more preferably monocyclic six-membered ring heteroaromatics having from one to three nitrogen atoms as heteroatoms, in particular pyridines, such as 2-ethylpyridine, 3-ethylpyridine, 4-ethylpyridine and 5-ethyl-2-methylpyridine, preferably 2-ethylpyridine and 5-ethyl-2-methylpyridine.

The process according to the invention may be carried out in one step, preferably in from two to six steps, more preferably in from two to four steps, in particular in two or three steps, isothermally, autothermally or adiabatically, preferably isothermally or adiabatically, more preferably adiabatically, in, for example, tube bundle reactors, tray reactors, shaft reactors, annular gap reactors or else in fluidized bed reactors, preferably in annular gap reactors or other reactor forms which allow a very low pressure drop of the catalyst bed, ore preferably in a battery of a plurality of such reactors attached in series, in particular arrangements having two or three such reactors attached in series.

The process according to the invention may be carried out oxidatively, i.e. in the presence of oxygen or air, or preferably nonoxidatively, i.e. in the absence of oxygen or air. Preference is given to adding no oxygen or air.

Useful catalysts for the process according to the invention are all known dehydrogenation catalysts, inter alia, those known from U.S. Pat. No. 5,689,023, U.S. Pat. No. 5,668,075, EP-A-1 027 928, EP-A-894 528, WO-A-99/49968, EP-A-297 685 and EP-A-305 020, in particular those which comprise iron oxide, potassium compound(s) and optionally promoters, for example, one or more compounds of cerium, molybdenum, tungsten, vanadium, calcium, magnesium, copper, titanium or chromium. Particular preference is given to using catalysts which comprise iron oxide, a potassium compound and also one or more compounds of cerium, magnesium, calcium and molybdenum. The weight ratio of potassium (calculated as K₂O) to iron oxide (calculated as Fe₂O₃) is generally from 0.01:1 to 2:1, preferably from 0.1:1 to 1:1. The catalysts preferably also comprise further promotors (calculated as oxides) in a weight ratio to iron oxide of from 0.01:1 to 1:1, preferably from 0.1:1 to 0.5:1. The catalysts are used as moldings or, when a fluidized bed reactor is used, in powder form. Preference is given to using the catalysts for fixed bed processes in extrudate form. Preference is given, for example, to using cylindrical extrudates having a diameter of from 1.5 to 10 mm, in particular having a diameter of from 2 to 6 mm and most preferably having a diameter of about 3 mm. Extruded catalysts having a star-shaped cross section as described in EP-A-1 027 928 or having a toothed wheel-shaped cross section as described in U.S. Pat. No. 5,097,091 are also suitable for the process according to the invention.

It is also possible to carry out the process according to the invention using dehydrogenation catalysts which are chromium-free. Chromium-free dehydrogenation catalysts are such catalysts which are prepared without the use of any chromium compound, and at most traces of chromium compounds are present in the catalyst feed stocks.

The suitable catalysts may also be regenerated. Depending on the catalysts used, regeneration may be carried out, for example, by introducing move steam, by introducing oxygen, while optionally interrupting the stream of the feed stock to be dehydrogenated. For example, regeneration of styrene catalysts may be achieved by increasing the steam/ethylbenzene ratio or interrupting the ethylbenzene introduction from time to time.

In industrial plants for preparing alkenylaromatic compounds, as are known, for example, from Encyclopedia of chemical processing and design, Marcel Dekker Inc., New York, John J. McKetta (Ed.), Vol 55, p. 201-204, the methane may be admixed at any one or at more than one point in the plant, but before contact with the catalyst. Preference is given to mixing the methane with the steam before entry into, for example, the steam superheater, and heating it together with the steam in the superheater.

The offgas obtained in the process according to the invention after removing the condensible constituents, comprising substantially methane, hydrogen and carbon dioxide, and also small amounts of carbon dioxide, ethane, ethene and higher hydrocarbons, may be used, for example, for heating purposes or as a feed stream for preparing synthesis gas.

The unsaturated compounds (alkenylaromatics) obtainable by the process according to the invention may, for example, be polymerized to give plastics or be used as building blocks for organic chemical syntheses.

### EXAMPLES

### Example 1

Preparation of the Catalyst

Iron oxide (type HP, Thyssen-Krupp) was continuously calcined for about 1 h in a rotary tube at 970° C. A slurry prepared by suspending 420 g of potash (potassium carbonate), 516 g of cerium carbonate hydrate (40% by weight cerium content), 74 g of ammonium heptamolybdate, 70 g of calcium carbonate, 55 g of magnesium hydroxycarbonate and 1880 g of the iron oxide in 4.5 l of water was processed to form a spray powder. The spray powder was pasted up by adding 1.5% by weight of potato starch with sufficient water (about 0.5 l) to form an extrudable mass, extruded to give extrudates having a 3 mm diameter, dried at 120° C., broken to a length of about 0.8 mm and finally calcined in a rotary tube at 800° C. for 1 h.

### Example 2

In an adiabatic two-stage pilot plant having an intermediate heater, ethylbenzene and water were mixed in the vapor phase with methane and passed over the catalyst from example 1 at an LHSV (=liquid hourly space velocity) of 0.48/h at a temperature of 630° C. and a pressure of 0.4 bar.

### Example 3 (noninventive)

Example 2 was repeated, except that operation was effected in the absence of methane.

The results from examples 2 and 3 are summarized in table 1.

TABLE 1

| | | | | | Spec. steam | Relative |
|---|---|---|---|---|---|---|
| | | | | Ethylbenzene | consumption, | space-time |
| Exam- | Steam/ethyl- | Methane/ethyl | Styrene | conversion | steam/styrene | yield |
| ple | benzene* | benzene* | selectivity | [%] | [kg/kg] | [%] |
| 2 | 5.9:1 | 1:1 | 96.8 | 66.4 | 1.59:1 | 107.4 |
| 3 | 5.9:1 | 0:1 | 96.2 | 62.2 | 1.71:1 | 100 |

*Molar ratio

1. A process for preparing alkenylaromatic compounds by reacting alkylaromatic compounds in the presence of steam and natural gas or methane over a suitable catalyst at a temperature of from 400 to 800° C. and a pressure of from 0.01 to 10 bar, wherein the molar ratio of steam to alkylaromatic compound is from 6.9:1 to 1:1, the molar ratio of methane to alkylaromatic compound is from 0.1:1 to 8:1, and the methane is admixed before contact with the catalyst.

2. A process for preparing alkenylaromatic compounds as claimed in claim 1, wherein the molar ratio of steam to alkylaromatic compound is from 5.95:1 to 2.5:1.

3. A process for preparing alkenylaromatic compounds as claimed in claim 1, wherein the molar ratio of steam to alkylaromatic compound is from 0.2:1 to 6:1.

4. A process for preparing alkenylaromatic as claimed in claim 1, wherein the reaction is carried out in from two to six steps.

5. A process for preparing alkenylaromatic compounds as claimed in claim 1, wherein the alkylaromatic compound used is isopropylbenzene, ethylbenzene, 1,2-diphenylethane or an alkylpyridine compound.

6. A process for preparing alkenylaromatic compounds as claimed in claim 1, wherein the hydrocarbon compound used is ethylbenzene.

7. A process for preparing alkenylaromatic compounds as claimed in claim 1, wherein the natural gas comprises at least 90 vol % of methane.

8. A process for preparing alkenylaromatic compounds as claimed in claim 1, wherein the reaction is carried out in radial flow reactors
